(19) United States
(12) Patent Application Publication
(10) Pub. No.: US 2022/0226169 A1
(43) Pub. Date: Jul. 21, 2022

(54) ABSORBENT ARTICLE

(71) Applicant: LIVEDO CORPORATION, Ehime (JP)

(72) Inventors: Kenji NAKAOKA, Tokushima (JP); Yuki TAKAHASHI, Tokushima (JP); Shingo TAKEDA, Tokushima (JP); Hiroaki TAKAKI, Osaka (JP); Takashi IZOE, Tokushima (JP)

(73) Assignee: LIVEDO CORPORATION, Ehime (JP)

(21) Appl. No.: 17/615,378

(22) PCT Filed: Apr. 21, 2020
(86) PCT No.: PCT/JP2020/017195

§ 371 Date: Nov. 30, 2021

(30) Foreign Application Priority Data
Jun. 7, 2019 (JP) 2019-107301

(51) Int. Cl. A61F 13/539; A61F 13/511

(52) U.S. Cl. CPC A61F 13/539; A61F 13/51104

(57) ABSTRACT

An absorbent article (1) comprising a top sheet (2), a back sheet (3) and an absorbent body (11) provided therebetween, wherein: the absorbent body (11) comprises plural layers of a sheet member and has a sealed part (14) in which the sheet members are bonded to each other and a non-sealed part (15) in which the sheet members are not bonded to each other, and an absorbent material is disposed in the non-sealed part (15), thereby forming an absorption area (16); the absorbent material disposed in the absorption area (16) contains an absorbent polymer in a proportion of 50 mass % or more the absorption area (16) is provided in a stripe shape extending in the front-rear direction; concave grooves (17) are formed from the top sheet (2) to the absorbent body (11) on one side and the other side in the width direction on a skin facing side of the absorbent article (1); and the concave grooves (17) are arranged such that the entire concave groove (17) overlaps with the absorption area (16) and end portions in the front-rear direction of the concave groove (17) are curved inward in the width direction.

### TECHNICAL FIELD

The present invention relates to an absorbent article such as an urine pad (including an incontinence pad), a sanitary napkin and a disposable diaper.

### BACKGROUND ART

Conventionally, absorbent articles comprising a top sheet, a back sheet and an absorbent body provided therebetween, in which a concave groove is formed from the top sheet to the absorbent body on a skin facing side of the absorbent article, are known (For example, Patent Literatures 1 to 3).

### CITATION LIST

### Patent Literature

PATENT LITERATURE 1

Japanese Unexamined Laid-open Patent Application Publication No. 2014-136126
PATENT LITERATURE 2

Japanese Unexamined Laid-open Patent Application Publication No. 2016-54755
PATENT LITERATURE 3

Japanese Unexamined Laid-open Patent Application Publication No. 2017-99563

### SUMMARY OF INVENTION

### Technical Problem

In absorbent articles, it is required that urine or the like excreted from a wearer is surely absorbed by and fixed to an absorbent body without occurring lateral leakage. Pulp fibers and absorbent polymers are often used as an absorbent material to be provided in the absorbent body, and when a proportion of the content of an absorbent polymer in the absorbent body is high, thickness of the absorbent body can be reduced and absorption capacity can be enhanced; however, absorption speed of urine or the like tends to be slower since an absorbent polymer has a weaker drawing force of urine or the like than pulp fibers. Therefore, in absorbent articles provided with the absorbent body having a high content of an absorbent polymer, it is particularly desired that the effect of preventing lateral leakage of urine or the like is ensured and the absorbent body absorbs more promptly.

The present invention has been made in view of the above circumstances, and an object of the present invention is to provide an absorbent article comprising an absorbent body containing an absorbent polymer as a main component of an absorbent material, that is excellent in absorbency of urine or the like and leakage prevention effect.

### Solution to Problem

The absorbent article of the present invention which solves the above problem is an absorbent article having a front-rear direction and a width direction, and comprising a top sheet, a back sheet and an absorbent body provided therebetween, wherein; the absorbent body comprises plural layers of a sheet member and has a sealed part in which the sheet members are bonded to each other and a non-sealed part in which the sheet members are not bonded to each other, and an absorbent material is disposed in the non-sealed part, thereby forming an absorption area; the absorbent material disposed in the absorption area contains an absorbent polymer in a proportion of 50 mass % or more; the absorption area is provided in a stripe shape extending in the front-rear direction; concave grooves extending continuously or intermittently in the front-rear direction are formed from the top sheet to the absorbent body on one side and the other side in the width direction on a skin facing side of the absorbent article; and the concave grooves are arranged such that the entire concave groove overlaps with the absorption area and end portions in the front-rear direction of the concave groove are curved inward in the width direction.

In the absorbent article of the present invention, since the concave grooves extending continuously or intermittently in the front-rear direction are formed from the top sheet to the absorbent body on one side and the other side in the width direction on a skin facing side of the absorbent article and the concave grooves provided so as to overlap with the absorption area, urine or the like excreted on the skin facing side of the absorbent article is suppressed from diffusing outward in the width direction beyond the concave grooves, whereby it becomes easy to prevent lateral leakage of urine or the like, as well as urine or the like easily permeates into the inside of the absorption area in the concave groove. In addition, since the concave groove is formed such that end portions in the front-rear direction thereof are curved inward in the width direction, urine or the like diffused in the front-rear direction in the concave groove flows inward in the width direction at the end portions in the front-rear direction of the concave groove, and leakage of urine or the like is less likely to occur at front and rear ends of the absorbent article. The urine or the like that has flowed inward in the width direction at the end portions in the front-rear direction of the concave groove reaches the sealed part of the absorbent body and then easily diffuses along the sealed part. As a result, urine or the like is easily absorbed from lateral side of the absorption area in the width direction, while being also absorbed from the skin facing side of the absorption area in the concave groove, whereby the whole of the absorption area is likely to contribute to absorption and fixation of urine or the like. Therefore, the absorbable article of the present invention is excellent in absorbability of urine or the like and leakage prevention effect.

The effect of providing the concave groove in the above manner becomes particularly remarkable as proportion of the content of an absorbent polymer in the absorbent material disposed in the absorption area is higher. For example, the higher the proportion of the content of an absorbent polymer in the absorbent material disposed in the absorption area is, the higher diffusibility of urine or the like on the skin facing side of the absorbent article becomes, and the effect of preventing lateral leakage by the concave groove is enhanced. In addition, there is concern that when an absorbent polymer absorbs urine or the like, the absorbent polymer swells and forms gels, and the absorbent polymer existing inside may not contribute to absorption of urine or the like; however, since the concave groove is formed from the top sheet to the absorbent body, urine or the like easily permeates into the inside of the absorbent body, and more amount of absorbent polymers easily contributes to the absorption of urine or the like. Further, by increasing the proportion of the content of an absorbent polymer in the absorbent material, the effect of diffusing urine or the like in the concave groove in the front-rear direction y is also enhanced, and the effect of forming, the end portion of the concave groove in the front-rear direction y to be curved inward in the width direction also comes to be more remarkable. From these points of view, it is preferable that the absorbent material disposed in the absorption area contains only an absorbent polymer.

It is preferable that the absorbent material is present at a bottom of the concave groove via the sheet member. By forming the concave groove in this manner, urine or the like in the concave groove is absorbed from both a lateral surface and a bottom surface of the concave groove, and urine or the like tends to be absorbed promptly.

It is preferable that the concave grooves extend intermittently in the front-rear direction. Thereby, diffusion speed of urine or the like in the concave groove in the front-rear direction is controlled, and leakage of urine or the like is less likely to occur at front and rear ends of the absorbent article.

It is preferable that the absorbent body is bonded to the top sheet at the non-sealed part and is not bonded to the top sheet at the sealed part. When the absorbent body is bonded to the top sheet in this manner, urine or the like can easily move from the top sheet to the inside of the absorbent body at the non-sealed part, namely, the absorption area. Meanwhile, at the sealed part, urine or the like that has passed through the top sheet tends to diffuse in the front-rear direction in a space between the top sheet and the absorbent, body. Therefore, absorbency and diffusibility of urine or the like can be enhanced.

It is preferable that the top sheet is made of an embossed nonwoven fabric having a linear embossed part. When the top sheet is configured in this manner, urine or the like easily diffuses along the linear embossed part on the top sheet. Meanwhile, on a non-embossed part, the top sheet is not compressed, and permeability of urine or the like through the top sheet is ensured.

It is preferable that the embossed nonwoven fabric has a quadrangular lattice-patterned embossed part composed of a plurality of straight line portions inclined in one direction and another direction with respect to the front-rear direction. When the top sheet is configured in this manner, urine or the like diffused along the linear embossed part can easily spread in multiple directions at lattice points of the quadrangular lattice-patterned embossed part, and so diffusibility of urine or the like in a plane direction on the top sheet is enhanced.

It is preferable that the straight line portion constituting the embossed parts are inclined clockwise or counterclockwise at an angle of 15° or more and 40° or less with respect to the front-rear direction. By arranging the straight line portion in this manner, urine or the like is more likely to diffuse in the front-rear direction than in the width direction on the top sheet.

It is preferable that the top sheet has a non-embossed part surrounded by the embossed part, and length of the non-embossed part in the width direction is longer than length of the sealed part in the width direction. When the non-embossed part is formed in this manner, at least a part of the each non-embossed part comes to be present so as to overlap with the absorption area. Therefore, in any of the non-embossed parts provided in the region overlapping with the absorbent body, urine or the like that has passed through the non-embossed part is easily absorbed by the absorbent material disposed in the absorption area promptly.

It is preferable that the embossed nonwoven fabric is composed of a short-fiber nonwoven fabric, and constituent fibers of the nonwoven fabric are oriented in the front-rear direction. Thereby, urine or the like is likely to preferentially diffuse in the front-rear direction on the top sheet while diffusibility in the width direction xis also ensured.

### Advantageous Effects of Invention

The absorbable article of the present invention is excellent in absorbability of urine or the like and an effect of preventing leakage.

### BRIEF DESCRIPTION OF THE DRAWINGS

FIG. 1 shows, an example of an absorbent article of the present invention, and shows a plan view of the absorbent article as viewed from a skin facing side thereof.

FIG. 2 shows a cross-sectional view taken along a line II-II of the absorbent article shown in FIG. 1.

FIG. 3 shows a cross-sectional view in a width direction of an absorbent body provided in the absorbent article shown in FIG. 2.

FIG. 4 shows another example of an absorbent article of the present invention, and shows a plan view of the absorbent article as viewed from a skin facing side thereof.

### DESCRIPTION OF THE PREFERRED EMBODIMENTS

An absorbent article of the present invention is explained with reference to drawings. However, the present invention is not limited to the embodiments shown in the drawings.

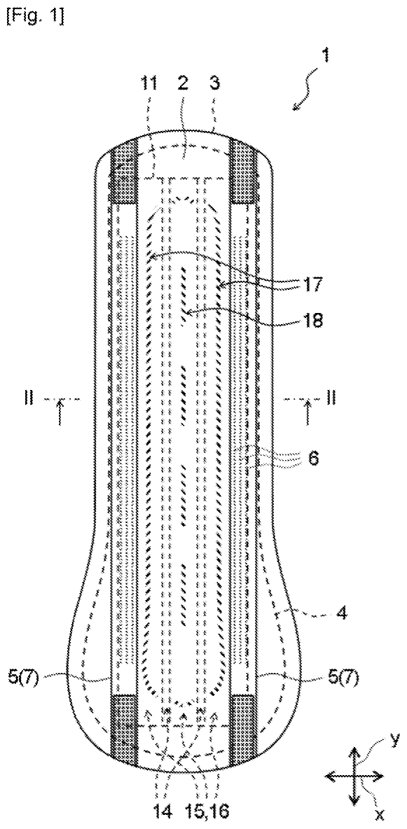

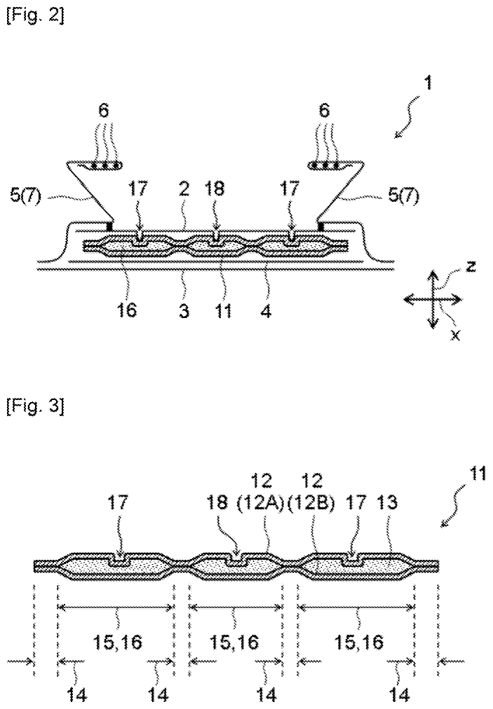

FIGS. 1 to 3 show a configuration example of an incontinence pad as an example of an absorbent article of the present invention. FIG. 1 shows a plan view of the absorbent article (incontinence pad) as viewed from a top sheet side, FIG. 2 shows a cross-sectional view taken along a line II-II of the absorbent article shown in FIG. 1, and FIG. 3 shows a cross-sectional view in a width direction of an absorbent body provided in the absorbent article shown in FIG. 2. In the drawings of the present application, an arrow x represents a width direction, an arrow y represents a front-rear direction, and a direction perpendicular to a plane formed by the arrows x and y represents a thickness direction z. In FIG. 1, an upper side of the drawing corresponds to a front side of the absorbent article, and a lower side of the drawing corresponds to a rear side of the absorbent article.

An absorbent article 1 comprises a top sheet 2, a back sheet 3 and an absorbent body 11 provided therebetween. The top sheet 2 is disposed on a skin facing side of the absorbent body 11, and the back sheet 3 is disposed on a non-skin facing side of the absorbent body 11. Excrement that has passed through the top sheet 2 is stored by the absorbent body 11. The back sheet 3 prevents excrement from leaking to the outside.

The absorbent article 1 has a front-rear direction y and a width direction x. The front-rear direction y means a direction extending in a front-rear direction at a crotch of a wearer when the absorbent article is worn. The width direction x means a direction orthogonal to the front-rear direction y on the same plane as the absorbent article, and corresponds to a left-right direction of a wearer when the absorbent article is worn. In addition, a skin facing side means a side facing a wearer's skin and a non-skin facing side means an opposite side of that when a wearer wears the absorbent article.

The top sheet 2 is disposed on a skin facing side of the absorbent body 11 and is provided so as to face a wearer's skin when the absorbent article is worn. The top sheet 2 is preferably liquid permeable. As the top sheet 2, a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof: or the like can be used, for example. As the top sheet 2, a woven fabric, a knitted fabric, a plastic film having holes or the like may be also used.

The back sheet 3 is disposed on a non-skin facing side of the absorbent body 11 and is preferably liquid impermeable As the back sheet 3, a nonwoven fabric made from hydrophobic fibers such as polyolefin (e.g., polypropylene and polyethylene), polyester (e.g., PET) and polyimide (e.g., nylon); a plastic film; or the like can be used, for example. As the back sheet 3, a laminate of a nonwoven fabric and a plastic be used.

In the drawings, a base sheet 4 is provided between the back sheet 3 and the absorbent body 11, and the base sheet 4 can improve shape-retaining property of the absorbent article 1. The base sheet 4 can be made of crepe paper or a nonwoven fabric. The base sheet 4 may not be provided.

In the case where a nonwoven fabric is used for the top sheet 2 or the back sheet 3, a spunbonded nonwoven fabric, an air-through nonwoven fabric, a point-bonded nonwoven fabric, a meltblown nonwoven fabric, an airlaid nonwoven fabric, an SMS nonwoven fabric or the like is preferably used as the nonwoven fabric.

It is preferable that rising flaps 5 are provided on both sides in the width direction x of the top sheet 2. The rising flap 5 prevents lateral leakage of urine or the like. The rising flap 5 can be made of a sheet material that can be used for the back sheet 3, and is preferably made of a liquid-impermeable plastic film or a liquid-impermeable nonwoven fabric. It is preferable that the rising flap 5 has a front end portion, a rear end portion and a middle portion between them in the front-rear direction y, the front end portion and the rear end portion are directly or indirectly joined, to the top sheet 2, and a flap elastic member 6 extending in the front-rear direction y is provided in the middle portion. By constituting the rising flap 5 in this manner, the middle portion is formed to be capable of standing up. In FIG. 1, the front end portion and the rear end portion of the rising flap 5 are embossed and joined to the top sheet 2 at the embossed part.

The rising flap 5 has a base part as a rising start point and a free end as a rising tip, and the flap elastic member 6 is disposed between the base part and the free end. The flap elastic member 6 is preferably fixed to the rising flap 5 with an adhesive or the like in a stretched state, and contraction force of the flap elastic member 6 promotes standing of the rising flap 5. The rising flap 5 can formed by joining side sheets 7 which extend in the front-rear direction y to both sides in the width direction x of the top sheet 2, and providing the flap elastic members 6 on an inner portion in the width direction x of the side sheet 7.

In the absorbent article 1 shown in the drawings, the side sheet 7 is folded in a Z-shape in a cross section in the width direction x, and the flap elastic member 6 is attached to a plane including the free end of the folded side sheet 7. That is, the rising flap 5 is folded along a folding line extending in the front-rear direction y between the base part and the free end, and the flap elastic member 6 is provided between the folding line and the free end. At the front end portion and the rear end portion of the rising flap 5, respective planes of the Z-shape folded side sheet 7 are joined to each other, whereby the front end portion and the rear end portion are indirectly fixed to the top sheet 2 (namely, via the side sheet 7). In addition, the Z-shape folding is preferably maintained by joining the respective planes of the side sheet 7 to each other at the front end portion and the rear end portion in this manner. In the absorbent article 1 shown in the drawings, a part on the base side of the folding line and a part on the free end side of the folding line of the rising flap 5 are embossed together at the front end portion and the rear end portion, whereby respective planes of the Z-shape folded side sheet 7 are joined to each other.

As shown in FIG. 3, the absorbent body 11 comprises plural layers of a sheet member 12, and has a sealed part 14 in which the sheet members 12 are bonded to each other and a non-sealed part 15 in which the sheet members 12 are not bonded to each other. An absorbent material 13 is disposed in the non-sealed part 15, thereby forming an absorption area 16. The absorbent material 13 disposed in the absorption area 16 contains an absorbent polymer in a proportion of 50 mass % or more. Since the absorption area 16 is formed in this manner, the absorbent article 1 can be configured so that absorption capacity of the absorbent body 11 is made high or the thickness of the absorbent body 11 is formed thin while securing the absorption capacity. In addition, diffusibility of urine or the like in the front-rear direction y in the absorbent body 11 can be enhanced by the sealed part 14.

The sheet member 12 includes a sheet member 12A located on a skin facing side and a sheet member 12B located on a non-skin facing side, and the absorbent material 13 is disposed between the sheet member 12A and the sheet member 12B. The sheet member 12A and the sheet member 12B may be provided separately, or one sheet member 12 may be folded back to give the sheet member 12A located on the skin facing side and the sheet member 12B located on the non-skin facing side.

At least a part of the sheet member 12 is formed to be liquid permeable, Preferably, at least the sheet member 12A on the skin facing side is formed to be liq id permeable, and more preferably, both the sheet member 12A on the skin facing side and the sheet member 12B on the non-skin facing side are formed to be liquid permeable. The liquid permeable sheet member 12 can be composed of, for example, a nonwoven fabric made from hydrophilic fibers such as cellulose, rayon and cotton, or a nonwoven fabric which is formed from hydrophobic fibers such as polyolefin (e.g., polypropylene and polyethylene), polyester (e.g PET) and polyamide (e.g., nylon), and in which the hydrophobic fibers are hydrophilized with a surfactant on the surface thereof. In addition, as the sheet member 12, a woven fabric, a knitted fabric, a plastic film having holes or the like may be used. Preferably, a sheet member made of a nonwoven fabric, that is, a nonwoven fabric sheet is used as the sheet member 12.

The sealed part 14 can be formed by joining the sheet members 12 to each other with an adhesive or by welding (heat sealing, ultrasonic welding or the like) them. The non-sealed part 15 is defined as a part where the sheet members 12 are not joined to each other, and a part other than the sealed part 14 corresponds to the non-sealed part 15. In the absorbent body 11, as long as the sheet members 12 are bonded to each other, an absorbent material may also be disposed in the sealed part 14.

The absorbent material 13 disposed in the absorption area 16 essentially contains an absorbent polymer. As the absorbent polymer, a polyacrylic absorbent polymer such as sodium polyacrylate; a starch absorbent polymer such as starch-acrylonitrile graft copolymer, starch-acrylic acid graft copolymer and starch-acrylamide graft copolymer; a polyvinyl alcohol absorbent polymer such as crosslinked polyvinyl alcohol: or the like can be used. Among them, a polyacrylic absorbent polymer such as sodium polyacrylate is preferably used, in view of high liquid absorption ability. The absorbent material 13 may include a hydrophilic fiber such as a cellulose fiber and a rayon fiber, or a thermal fusion fiber such as a polyolefin (e.g., polyethylene and polypropylene) fiber, a polyester (e.g., PET) fiber and a polyamide fiber. The thermal fusion fibers is preferably hydrophilized with a surfactant or the like to increase affinity with urine or the like.

The proportion of the content of an absorbent polymer contained in the absorbent material 13 is 50 mass % or more, preferably 70 mass % or more, more preferably 80 mass % or more, and even preferably 90 mass % or more. By increasing the proportion of the content of an absorbent polymer contained in the absorbent material 13, absorbent body 11 can be formed thin while having a high absorption capacity.

A shape (planar shape) of the absorbent body 11 is not particularly limited. Examples of the shape of the absorbent body 11 include a substantially rectangular shape, an hourglass shape, a gourd shape, a battledore shape, and others.

In the absorbent body 11, the absorption area 16 is provided in a stripe shape extending in the front-mar direction y. That is, at least two absorption areas 16 are provided side by side in the width direction x, and preferably three or more absorption areas 16 are provided side by side in the width direction x. Meanwhile, preferably eight or less, more preferably six or less absorption areas 16 are provided side by side in the width direction x. Each absorption area 16 is preferably provided so as to extend over the entire absorbent body 11 in the front-rear direction y. In the absorbent article 1 shown in the drawings, three absorption areas 16 are provided side by side in the width direction x.

It is preferable that the sealed part 14 is also provided in a stripe shape extending in the front-rear direction y, and the sealed parts 14 are preferably provided so as to extend in the front-rear direction y at both one end part and the other end par in the width direction x of the absorbent body 11 respectively. By providing the sealed part 14 in this manner, urine or the like is easily diffused on the absorbent body 11 in the front-rear direction y, and urine or the like is easily absorbed by the absorbent body 11 promptly.

Length of each sealed part 14 in the width direction x is preferably 1 mm or longer, more preferably 2 mm or longer, and preferably 10 mm or shorter, more preferably 8 mm or shorter. Length of each absorption area 16 (or non-sealed part 15) in the width direction x is preferably 5 mm or longer, more preferably 8 mm or longer, and preferably 50 mm or shorter, more preferably 30 mm or shorter, even more preferably 20 mm or shorter. The ratio of the length of the sealed part 14 to the length of the absorption area 16 (or the non-sealed part 15) in the width direction x is preferably 5/95 or more, more preferably 10/90 or more, even more preferably 15/85 or more, and preferably 50/50 or less, more preferably 60/40 or less, as a ration of the length of the sealed part 14 in the width direction x/the length of the absorption area 16 (or the non-sealed part 15).

Concave grooves 17 extending continuously or intermittently in the front-rear direction y are formed from the top sheet 2 to the absorbent body 11 on one side and the other side in the width direction x on the skin facing side of the absorbent article 1. Since the top sheet 2 and the absorbent body 11 are integrally recessed in the concave groove 17, urine or the like that has passed through the top sheet 2 easily permeates into the inside of the absorption area 16 in the concave groove 17. Further, by forming the concave groove 17 on one side and the other side in the width direction x on the skin facing side of the absorbent article 1, urine or the like excreted on the skin facing side of the absorbent article 1 is suppressed from diffusing outward in the width direction x beyond the concave groove 17, and it becomes easy to prevent lateral leakage of urine or the like. The urine or the like that has reached the concave groove 17 is likely to diffuse in the front-rear direction y in the concave groove 17, whereby a wider range of the absorbent body 11 in the front-rear direction y comes to contribute absorption of urine or the like.

The concave grooves 17 are arranged such that the entire concave groove 17 overlaps with the absorption area 16 and end portions in the front-rear direction y of the concave groove 17 is curved inward in the width direction x. It is preferable that both an inner edge and an outer edge, with respect to the width direction x, of the concave groove 17 are curved inward in the width direction x at the end portions in the front-rear direction y. As the concave grooves 17 are arranged in this manner, urine or the like diffused in the front-rear direction y in the concave groove 17 flows inward in the width direction x at the end portions in the front-rear direction y of the concave groove 17, and leakage of urine or the like is less likely to occur at front and rear ends of the absorbent article 1. The urine or the like that has flowed inward in the width direction x at the end portions in the front-rear direction y of the concave groove 17 reaches the sealed part 14 of the absorbent body 11 and then easily diffuses along the sealed part 14. As a result, urine or the like is easily absorbed from lateral side of the absorption area 16 in the width direction x, while being also absorbed from the skin facing side of the absorption area 16 in the concave groove 17, whereby the whole of the absorption area 16 is likely to contribute to absorption and fixation of urine or the like. Therefore, the absorbable article 1 is excellent in absorbability of urine or the like and leakage prevention effect.

The effect of providing the concave groove 17 in the above manner becomes particularly remarkable as the proportion of the content of an absorbent polymer in the absorbent material 13 disposed in the absorption area 16 is higher. An absorbent polymer has a weaker drawing force of urine or the like relative to a hydrophilic fiber such as a pulp fiber, and tends to have slower absorption speed of urine or the like. Therefore, the higher the proportion of the content of an absorbent polymer in the absorbent material 13 disposed in the absorption area 16 is, the higher diffusibility of urine or the like on the skin facing side of the absorbent article 1 becomes, and the effect of preventing lateral leakage by the concave groove 17 is enhanced. In addition, there is concern that when an absorbent polymer absorbs urine or the like, the absorbent polymer swells and forms gels, and the absorbent polymer existing inside may not contribute to absorption of urine or the like; however, since the concave groove 17 is formed from the top sheet 2 to the absorbent body 11, urine or the like easily permeates into the inside of the absorbent body 11 (particularly, the inside of the absorption area 16 in the thickness direction), and more amount of absorbent polymers easily contribute to the absorption of urine or the like. Further, by increasing the proportion of the content of an absorbent polymer in the absorbent material 13, the effect of diffusing urine or the like in the concave groove 17 in the front-rear direction y is also enhanced, and the effect of forming the end portions of the concave groove 17 in the front-rear direction y to be curved inward in the width direction x also comes to be more remarkable. From these points of view, the proportion of the absorbent polymer contained in the absorbent material 13 is preferably 95 mass % or more, and it is particularly preferable that only an absorbent polymer is disposed in the absorption area 16 as the absorbent material 13.

It is preferable that the concave grooves 17 are provided so as to overlap with the absorption area 16 closest to one end in the width direction x of the absorbent body 11 and the absorption area 16 closest to the other end in the width direction x of the absorbent body 11, respectively. That is, it is preferable that the concave groove 17 provided on one side in the width direction x is arranged so as to overlap with the absorption area 16 closest to one end in the width direction x of the absorbent body 11 and the concave groove 17 provided on the other side in the width direction x is arranged so as to overlap with the absorption area 16 closest to the other end in the width direction x of the absorbent body 11. By providing the concave groove 17 in this manner, urine or the like excreted from a wearer can easily diffuse on the top sheet 2 over a wide range in the width direction x.

Provided that, as a relative position of the absorbent body 11 in the front-rear direction y, a front end, thereof is set to be 0% and a rear end thereof is set to be 100%, a front end of the concave groove 17 is preferably located at a position of 3% to 30%, more preferably at a position of 5% to 25%, and a rear end of the concave groove 17 is preferably located at a position of 70% to 97%, more preferably at a position of 75% to 95%. By providing the concave groove 17 in this manner, it is possible to enhance the effect of preventing lateral leakage of urine or the like in a wider range in the front-rear direction y.

It is preferable that the front end and the rear end of the concave groove 17 are located near a rear end of the front end portion of the rising flap 5 or a front end of the rear end portion of the rising flap 5, where the front end portion and the rear end portion are directly or indirectly joined to the top sheet 2. Specifically, the front end of the concave groove 17 is preferably located within a range of 15 mm forward and 30 mm rearward, more preferably within a range of 10 mm forward and 20 mm rearward from the rear end of the front end portion of the rising flap 5. The rear end of the concave groove 17 is preferably located within a range of 30 mm forward and 15 mm rearward, more preferably within a range of 20 mm forward and 10 mm rearward from the front end of the rear end portion of the rising flap 5. In this case, since the concave groove 17 is provided so as to substantially overlap with the middle portion of the rising flap 5 in the front-rear direction y, the effect of preventing lateral leakage of urine or the like is further enhanced.

The concave groove 17 s preferably provided so as to extend intermittently in the front-rear direction y Thereby, diffusion speed of urine or the like in the concave groove 17 in the front-rear direction y is controlled, and leakage of urine or the like is less likely to occur at front and rear ends of the absorbent article 1. In this case, an intermittent distance of the concave groove 17 (the shortest separation distance between the adjacent concave grooves 17 in the front-rear direction y) is preferably 0.5 mm or longer, more preferably 1 mm or longer, and preferably 5 mm or shorter, more preferably 3 mm or shorter. The intermittent portion of the concave groove 17 is preferably formed so as to extend obliquely with respect to the width direction x, and as a result, urine or the like is less likely to move outward in the width direction x beyond the concave groove 17 even when the concave groove 17 is provided intermittently.

It is preferable that the absorbent material 13 is present at a bottom of the concave groove 17 via the sheet member 12. That is, the concave groove 17 is formed to be recessed on the skin facing side of the absorption area 16, and it is preferable that the concave groove 17 is not formed so as to penetrate the absorbent material 13 disposed in the absorption area 16 in the thickness direction but the absorbent material 13 is present at a bottom of the 17 via the sheet member 12. By forming the concave groove 17 in this manner, urine or the like in the concave groove 17 is absorbed from both a lateral surface and a bottom surface of the concave groove 17, and urine or the like tends to be absorbed promptly. More preferably, an absorbent polymer is present at the bottom of the groove 17 via the sheet member 12.

The absorbent article 1 may be provided with a concave groove 18 extending in the front-rear direction y at a center in the width direction x on the skin facing side thereof. Also in this case, it is preferable that the entire concave groove 18 is arranged so as to overlap with the absorption area 16. In addition, it is preferable that the concave groove 18 is provided intermittently in the front-rear direction y. The concave groove 18 provided at a center in the width direction x is preferably provided so as to extend in the width direction x at the front-rear end thereof, thereby leakage of urine or the like in the front-rear direction y tends to be prevented.

It is preferable that the absorbent body 11 is bonded to the top sheet 2 at the non-sealed part 15 and is not bonded to the top sheet 2 at the sealed part 14. When the absorbent body 11 is bonded to the top sheet 2 in this manner, urine or the like can easily move from the top sheet 2 to the inside of the absorbent body 11 at the non-sealed part 15, namely, the absorption area 16. Meanwhile, at the sealed part 14, urine or the like that has passed through the top sheet 2 tends to diffuse in the front-rear direction y in a space between the top sheet 2 and the absorbent body 11. Therefore, the absorbent article 1 can be made to be excellent in absorbency and diffusibility of urine or the like.

It is preferable that the non-sealed part 15 of the absorbent body 11 is bonded to the top sheet 2 with an adhesive. In this case, the adhesive is preferably applied in a linear, net-like or scattered spot pattern from the viewpoint that urine or the like that has passed through the top sheet 2 is promptly absorbed to the absorbent body 11 at the absorption area 16. As such a method of applying the adhesive, an adhesive layer for joining the top sheet 2 and the absorbent body 11 is preferably formed by a bead method, a curtain spray method, an omega coating method, a spiral coating method, a pattern coating method or the like.

It is preferable that the sealed part 14 of the absorbent body 11 maintains the bonding between the sheet members 12 even when the absorbent material 13 absorbs. As a result, even after the absorbent material 13 absorbs, the ability of the absorbent body 11 to absorb urine or the like can be easily secured. From this point of view, the sealed part 14 is preferably formed by bonding the sheet members 12 to each other with an adhesive such as a rubber-based adhesive and a styrene-based elastomer or welding them to each other.

On the skin facing side of the absorbent body 11, that is, between the top sheet 2 and the absorbent body 11, a second sheet may be provided (not shown). The second sheet is used for promptly drawing urine or the like excreted on the skin facing side of the absorbent article 1 into the inside of the absorbent article 1 or promoting diffusion of urine or the like that has passed through the top sheet 2 in a plane direction. In the case where the second sheet is provided on the skin facing side of the absorbent body 11, it is preferable that the absorbent body 11 is bonded to the second sheet at the non-sealed part 15 and is not bonded to the second sheet at the sealed part 14. Further, the concave grooves 17 and 18 may be formed by integrally recessing the top sheet 2, the second sheet and the absorbent body 11.

On the non-skin facing side of the absorbent body 11, a separate second absorbent body may be provided (not shown). Examples of the second absorbent body include an aggregate of hydrophilic fibers such as pulp fibers, and the aggregate to which an absorbent polymer is mixed or scattered. Further, as the second absorbent body, a sheet-shaped absorbent body configured such that an absorbent polymer is disposed between sheet members but a pulp fiber is not disposed between them may be used. In view of forming the thinner absorbent article 1, it is preferable that the second absorbent body is not arranged.

It is preferable that the top sheet 2 is made of an embossed nonwoven fabric having a linear embossed part. When the top sheet 2 is configured in this manner, the top sheet 2 is compressed at the embossed part, and urine or the like easily diffuses along the linear embossed part on the top sheet 2. Meanwhile, on a non-embossed part, the top sheet 2 is not compressed, and permeability of urine or the like through the top sheet 2 is ensured.

The linear embossed part may be provided on the top sheet 2 at least at a part of a region overlapping with the absorbent body 11 in a certain pattern, and is preferably provided on the top sheet 2 over the entire region overlapping with the absorbent body 11. By providing the embossed part in this manner, urine or the like is likely to diffuse over a wide area on the top sheet 2, and the entire absorbent body 11 tends to contribute to absorption of urine or the like. More preferably, the embossed part is provided over the entire top sheet 2.

The linear embossed part may be provided in a straight line shape, may be provided in a curved shape, or may be provided in a combination thereof. The embossed part is preferably provided so as to promote diffusion of urine or the like in the front-rear direction y, and for example, it is preferably provided in a straight line shape extending in a direction of an angle of less than 45° clockwise or counterclockwise with respect to the front-rear direction y or in a meandering line shape. The angle is preferably 40° or less, more preferably 35° or less, and even more preferably 30° or less. Thereby, lateral leakage of urine or the like is less likely to occur.

The embossed part may be formed by pressing the top sheet 2 in a non-heated state, or may be formed by pressing the top sheet 2 in a heated state. In the latter case, the embossed part can be formed by heat sealing, ultrasonic welding, or the like. In either case, the embossed part is preferably formed by pressing the top sheet 2 alone. In view of increasing diffusibility of urine or the like at the embossed part, the embossed part is preferably formed by pressing in a heated state, that is, the embossed part is preferably a heat embossed part. In this case, the top sheet 2 is preferably made of a nonwoven fabric containing a thermal fusion fiber. Further, it is preferable that at least a part of constituent fibers of the nonwoven fabric is melted in the heat embossed part. Examples of the thermal fusion fiber include fibers composed of a thermoplastic resin such as polyolefin (e.g., polyethylene and polypropylene), polyester (e.g., PET) and polyamide (for example, nylon).

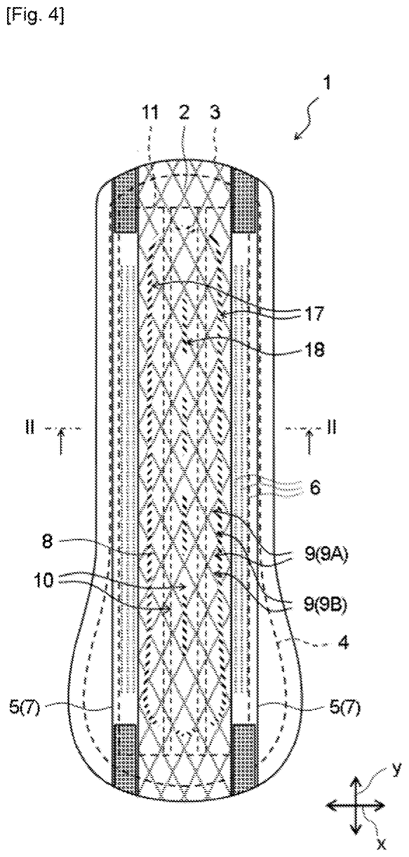

As shown in FIG. 4. the top sheet 2 is preferably made of an embossed nonwoven fabric having a quadrangular lattice-patterned embossed part 8 composed of a plurality of straight line portions inclined in one direction and anotherr direction with respect to the front-rear direction y. When the top sheet 2 is configured in this manner, urine or the like diffused along the linear embossed part 8 can easily spread in multiple directions at lattice points of the quadrangular lattice-patterned embossed part 8, and so diffusibility of urine or the like in the plane direction on the top sheet 2 is enhanced. Meanwhile, on the top sheet 2, a non-embossed part 10 is formed surrounded by the quadrangular lattice-patterned embossed part 8, and as a flow direction of urine or the like changes at the lattice points of the quadrangular lattice-patterned embossed part 8, urine or the like easily spreads to the non-embossed part 10, whereby urine or the like easily permeates the top sheet 2 at the non-embossed part 10 promptly.

The quadrangular lattice-patterned embossed part 8 is formed by intersecting a plurality of straight line portions 9 extending inclined in one direction with respect to the front-rear direction y (hereinafter, referred to as a “first straight line portion 9A”) and a plurality of straight line portions 9 extending inclined in another direction with respect to the front-rear direction y (hereinafter, referred to as a “second straight line portion 9B”). The plurality of first straight line portions 9A and the plurality of second straight line portions 9B are intersected with each other, whereby a plurality of substantially rhombus-shaped non-embossed parts 10 are preferably formed on the top sheet 2. The meaning of “substantially rhombus” includes not only a perfect rhombus but also a rhombus with rounded corners and a rhombus with cut comers For example. when the top sheet 2 is embossed in a quadrangular lattice pattern, the line width of the first straight line portion 9A and/or the second straight line portion 9B may be thicker at an intersection of the first straight line portion 9A and the second straight line portion 9B than that in other parts. In such a case, the non-embossed part 10 may be formed in a rhombus with rounded corners or a rhombus with cut corners. It is preferable that a plurality of substantially rhombus-shaped non-embossed parts 10 are arranged side by side both in the width direction x and the front-rear direction y.

The first straight line portions 9A are preferably inclined clockwise at an angle of 15° or more, more preferably 20° or more, and preferably 40° or less, more preferably 35° or less with respect to the front-rear direction y. The second straight line portions 9B are preferably inclined counterclockwise at an angle of 15° or more, more preferably 20° or more, and preferably 40° or less, more preferably 35° or less with respect to the front-rear direction y. By arranging the first straight line portions 9A and the second straight line portions 9B in this manner, urine or the like is more likely to diffuse in the front-rear direction y than in the width direction x on the top sheet 2.

The plurality of first straight line portions 9A are preferably arranged so as to be inclined in one direction at substantially equal pitches, and the plurality of second straight line portions 9B are preferably arranged so as to be inclined in another direction at substantially equal pitches. Pitch length of the plurality of first straight line portions 9A and pitch length of the plurality of second straight line portions 9B are preferably 3 mm or longer, more preferably 4 mm or longer, even more preferably 5 mm or longer, and preferably 15 mm or shorter, more preferably 12 mm or shorter, even more preferably 10 mm or shorter. The pitch length of the plurality of first straight line portions 9A (or second straight line portions 9B) means a distance between centers of line widths of the adjacent first straight line portions 9A (or second straight line portions 9B). The line width of the first straight line portion 9A and the second straight line portion 9B is preferably 0.5 mm or thicker, more preferably 0.8 mm or thicker, and preferably 2.5 mm or thinner, more preferably 2.0 mm or thinner. The pitch length of the plurality of first straight line portions 9A is preferably larger than the line width of the first straight line portion 9A, and for example, the pitch length of the plurality of first straight line portions 9A is preferably 2 times or more, more preferably 3 times or more, and preferably 20 times or less, more preferably 15 times or less, even more preferably 10 times or less of the line width of the first straight line portion 9A. The pitch length of the plurality of second straight line portions 9B is preferably larger than the line width of the second straight line portion 9B, and for example, the pitch length of the plurality of second straight line portions 9B is preferably 2 times or more, more preferably 3 times or more, and preferably 20 times or less, more preferably 15 times or less, even more preferably 10 times or less of the line width of the second straight line portion 9B.

The substantially rhombus-shaped non-embossed part 10 is preferably formed so that the length in the front-rear direction y is longer than the length in the width direction x. Specifically, it is preferable that each non-embossed part 10 surrounded by the quadrangular lattice-patterned embossed parts 8 is formed so that the length in the front-rear direction y is longer than the length in the width direction x. By forming the non-embossed part 10 in this manner, urine or the like is likely to diffuse in the front-rear direction y on the top sheet 2. The length of the non-embossed part 10 in the front-rear direction y is preferably 1.2 times or more, more preferably 1.5 times or more, and preferably 4.0 times or less, more preferably 3.0 times or less of the length of the non-embossed part 10 in the width direction x. When the non-embossed part 10 is formed in this manner, urine or the like is likely to preferentially diffuse in the front-rear direction y on the top sheet 2 while diffusibility in the width direction x is also ensured.

The length of the non-embossed part 10 in the front-rear direction y is, for example, preferably 5 mm or longer, more preferably 7 mm or longer, even more preferably 10 mm or longer, and preferably 25 mm or shorter, more preferably 22 mm or shorter, even more preferably 20 mm or shorter. The length of the non-embossed part 10 in the width direction x is, for example, preferably 3 mm or longer, more preferably 4 mm or longer, even more preferably 5 mm or longer, and preferably 16 mm or shorter, more preferably 14 mm or shorter, even more preferably 12 mm or shorter.

The length of the non-embossed part 10 in the width direction x is preferably longer than the length of the sealed part 14 of the absorbent body 11 in the width direction x. Specifically, it is preferable that the length in the width direction x of each non-embossed part 10 surrounded by the quadrangular lattice-patterned embossed parts 8 is longer than the length in the width direction x of the sealed part 14 of the absorbent body 11. When the non-embossed part 10 is formed in this manner, the entire non-embossed part 10 does not overlap with the sealed part 14 but only a part of the non-embossed part 10 overlaps with the absorption area 16, in the non-embossed part 10 provided so as to overlap with the sealed part 14. Therefore, in any of the non-embossed parts 10 provided in a region overlapping with the absorbent body 11, urine or the like that has passed through the non-embossed part 10 is easily absorbed by the absorbent material 13 disposed in the absorption area 16 promptly.

From the viewpoint that urine or the like easily diffuses in the front-rear direction y on the top sheet 2, it is preferable that the top sheet 2 is composed of a nonwoven fabric and constituent fibers of the nonwoven fabric are oriented in the front-rear direction y. More preferably, the top sheet 2 is made of a short-fiber nonwoven fabric, and the constituent fibers of the nonwoven fabric are oriented in the front-rear direction y. Thereby, urine or the like is likely to preferentially diffuse in the front-rear direction y on the top sheet 2 while diffusibility in the width direction x is also ensured.

Examples of the nonwoven fabric whose constituent fibers orient in one direction include a spunbonded nonwoven fabric, an air-through nonwoven fabric and a spun-lace nonwoven fabric. The orientation direction of constituent fibers of a nonwoven fabric can be determined by observing a surface of the nonwoven fabric with a microscope or the like. For example, in the spunbonded nonwoven fabric, the orientation direction of the constituent fibers is explained as follows. A spunbonded nonwoven fabric is formed by, for example, melting a polymer material, extruding it from a spinneret to be stretched, and collecting it on a conveyer belt or the like to form a web; and on this occasion, the web (fibers) collected on the conveyer belt is arranged along a traveling direction of the conveyer belt. Therefore, in this case, the web (fibers) comes to be oriented in the traveling direction (MD direction) of the conveyer belt In the air-through nonwoven fabric, the orientation direction of the constituent fibers can be arranged by appropriately setting a collection method of raw short fibers in forming a fiber aggregate or a fiber-opening method in forming a web, upon manufacturing the nonwoven fabric. In the spun-lace nonwoven fabric, the orientation direction of the constituent fibers can be arranged by controlling flow of water in which short fibers are dispersed and depositing, upon manufacturing the nonwoven fabric.

The top sheet 2 is preferably composed of an air-through nonwoven fabric or a spunlace nonwoven fabric as a short-fiber nonwoven fabric. Since the spunlace nonwoven fabric and the air-through nonwoven fabric can be formed with a relatively low bulk density, they have excellent permeability to urine or the like. Further, as described above, in the spunlace nonwoven fabric and the air-through nonwoven fabric, the constituent fibers of the nonwoven fabric are preferably oriented in the front-rear direction y, whereby the diffusibility in the front-rear direction y can be enhanced.

In the above, the absorbent article of the present invention is explained with reference to the drawings; however, the absorbent article of the present invention is not limited to an incontinence pad such as shown in the drawings, and may be a disposable diaper or a sanitary napkin.

In the case where the absorbent article is a disposable diaper, the disposable diaper is configured so as to have, for example, a front abdomen part, a rear back part, and a crotch part positioned therebetween and provided with an absorbent body. The front abdomen part is a part applied to an abdomen side of a wearer in wearing the disposable diaper, and the rear back part is a part applied to a back side of the wearer in wearing the disposable diaper. The crotch part is a part positioned between the front abdomen part and the rear back part and applied to a crotch of the wearer.

The disposable diaper may be a tape-type disposable diaper or may be a pants-type disposable diaper. The tape-type disposable diaper is provided with fastening members on both end parts in the width direction of the rear back part, for example, and can be formed into a pants shape by fastening the fastening members to the front abdomen part. The pants-type disposable diaper has a pants shape having a waist opening and a pair of leg openings, and is formed in a pants shape before wearing.

In the case where the absorbent article is a disposable diaper, the disposable diaper may be formed, for example, so that a laminate comprising a top sheet, a back sheet and an absorbent body provided therebetween has the front abdomen part, the rear back part and the crotch part positioned therebetween. The disposable diaper may also comprise an exterior member having the front abdomen part, the rear back part and the crotch part positioned therebetween, and an absorbent main body comprising a top sheet, a back sheet and an absorbent body provided therebetween, wherein the absorbent main body is disposed on the crotch part of the exterior member. In the latter case, by forming the exterior member in a pants shape, a pants-type disposable diaper can be obtained. In any case, the absorbent article of the present invention can be obtained by configuring the top sheet or the absorbent body of the absorbent article as described above,

This application claims priority to Japanese Patent Application No. 2019-107301, filed on Jun. 7, 2019. All of the contents of the Japanese Patent Application No. 2019-107301, filed on Jun. 7, 2019, are incorporated by reference herein.

### REFERENCE SIGNS IST

1: an absorbent article

2: a top sheet

3: a back sheet

4: a base sheet

5: a rising flap

6: a flap elastic member

7: a side sheet

8: an embossed part

9: a straight line portion, 9A: a first straight line portion, 9 a second straight line portion

10: a non-embossed part

11: an absorbent body

12: a sheet member

13: an absorbent material

14: a sealed part

15: a non-sealed part

16: an absorption area

17, 18: a concave groove

1. An absorbent article having a front-rear direction and a width direction, and comprising a top sheet, a back sheet and an absorbent body provided therebetween, wherein:
the absorbent body comprises plural layers of a sheet member and has a sealed part in which the sheet members are bonded to each other and a non-sealed part in which the sheet members are not bonded to each other, and an absorbent material is disposed in the non-sealed part, thereby forming an absorption area;
the absorbent material disposed in the absorption area contains an absorbent polymer in a proportion of 50 mass % or more;
the absorption area is provided in a stripe shape extending in the front-rear direction;
concave grooves extending continuously or intermittently in the front-rear direction are formed from the top sheet to the absorbent body on one side and the other side in the width direction on a skin facing side of the absorbent article; and
the concave grooves are arranged such that the entire concave groove overlaps with the absorption area and end portions in the front-rear direction of the concave groove are curved inward in the width direction.

2. The absorbent article according to claim 1, wherein the absorbent material disposed in the absorption area contains only an absorbent polymer.

3. The absorbent article according to claim 1, wherein the absorbent material is present at a bottom of the concave groove via the sheet member.

4. The absorbent article according to claim 1, wherein the concave grooves extend intermittently in the front-rear direction.

5. The absorbent article according to claim 1, wherein the absorbent body is bonded to the top sheet at the non-sealed part and is not bonded to the top sheet at the sealed part.

6. The absorbent article according to claim 1, wherein the top sheet is made of an embossed nonwoven fabric having a linear embossed part.

7. The absorbent article according to claim 6, wherein the embossed nonwoven fabric has a quadrangular lattice-patterned embossed part composed of a plurality of straight line portions inclined in one direction and another direction with respect to the front-rear direction.

8. The absorbent article according to claim 7, wherein the straight line portions constituting the embossed part are inclined clockwise or counterclockwise at an angle of 15° or more and 40° or less with respect to the front-rear direction.

9. The absorbent article according to claim 7, wherein the top sheet has a non-embossed part surrounded by the embossed part, and length of the non-embossed part in the width direction is longer than length of the sealed part in the width direction.

10. The absorbent article according to claim 6, wherein the embossed nonwoven fabric is composed of a short-fiber nonwoven fabric, and constituent fibers of the nonwoven fabric are oriented in the front-rear direction.
